(19) United States
(12) Patent Application Publication
(10) Pub. No.: US 2007/0213799 A1
(43) Pub. Date: Sep. 13, 2007

(54) Cochlear Implant Electrode Configuration for Drug Eluting

(76) Inventors: Claude Jolly, Innsbruck (AT); Guido Reetz, Innsbruck (AT); Mohammad Imani, Tehran (IR); Hamid Mirzadeh, Tehran (IR); Farhid Farahmand, Tehran (IR)

Correspondence Address: BROMBERG & SUNSTEIN LLP, 125 SUMMER STREET, BOSTON, MA, 02110-1618, US

(21) Appl. No.: 11/684,167

(22) Filed: Mar. 9, 2007

Related U.S. Application Data
(60) Provisional application No. 60/780,667, filed on Mar. 9, 2006.

(51) Int. Cl. A61N 1/05

(52) U.S. Cl. 607/137

(57) ABSTRACT

A cochlear electrode array for electrically stimulating cochlear tissues including a drug eluting portion will be disclosed. This device is adapted to release over time a therapeutically effective amount of a pharmaceutical agent for the inner ear. The pharmaceutical agent can be released locally for different therapeutic applications.

This application claims priority from U.S. Provisional Application 60/780,667, filed Mar. 9, 2006, the contents of which are incorporated herein by reference.

### FIELD OF THE INVENTION

The invention relates to a drug eluting cochlear implant electrode for the transient elution of pharmacologically active agents into the inner ear.

### BACKGROUND ART

Electrical stimulation of the inner ear has been very successful in restoring sound sensation to patients afflicted with deafness. Intra-cochlear electrodes are intended to restore some sense of hearing by direct electrical stimulation of the neural tissue in proximity of an electrode contact. The electrical stimulation is accomplished with an implanted cochlear implant stimulator connected to an electrode inserted deep into the scala tympani cavity.

But the insertion of the electrode causes a variable amount of trauma and connective tissue growth. The amount of trauma is very difficult to predict and depends on the cochlea anatomy, the electrode design and the insertion technique. The trauma inflicted to the tissues may subsequently cause apoptosis and/or necrosis of nervous tissue (i.e., hair cells and spiral ganglion cells). Tissue growth and trauma may limit the performance of the implant, and trauma to spiral ganglion cells is cumulative and cannot be undone in the present state of technology. As more patients with significant usable residual hearing receive a cochlear implant, it becomes ever more important to use a minimally traumatic electrode, and as more patients are implanted at a young age who will be re-implanted several times during their lifetime, each consecutive insertion should limit the trauma to spiral ganglion cells to a minimum.

Trauma is usually caused by the electrode insertion into the delicate tissue of the inner ear. Insertion requires mechanical forces to be applied on the electrode to overcome the friction of the electrode against the tissue of the spiraling cochlea. To reduce trauma to the organ or tissue, electrodes and catheters should be soft and flexible, and insertion forces should be minimum. Unfortunately, most cochlear implant electrodes on the market today require significant force to be inserted, even for distances which are much less than the full length of the scala tympani.

The force required to insert an electrode or catheter is related to its size, geometry, and fabrication material. Materials used in such devices include materials for wires, contacts, functional metallic or polymer segments, and bulk material. The size of the device, the rigidity of the material used, the hydrophobicity of the outer shell of the electrode array, the energy stored in one way or another on the electrode surface, and the insertion process of the device all have an impact on the amount and location of tissue damage that will be inflicted during electrode placement.

Damage and trauma cause bleeding, inflammation, perforation of the soft tissues, tears and holes in membranes, and fracture of thin osseous structures. The resulting damage may cause loss of surviving hair cells, retrograde degeneration of the dendrites which innervate the organ of Corti, and in the worst case, spiral ganglion cell death in the Rosenthal's canal. Cell death means that quantitatively less neural tissue is available for stimulation, and qualitatively that fewer frequency-tuned fibers are available to represent frequency information. Further loss of hair cells and loss of dendrites without loss of spiral ganglion cells means that acoustic stimulation is no longer possible, and that no synergetic effects between acoustic and electric stimulation will be available. Electro-acoustic synergetic effects may be important for good sound discrimination in noisy environments.

Another inconvenience with cochlear implants is the rise in measured electrode impedance post-surgery. This rise is thought to be caused by encapsulation of the electrode by a tight fibrous membrane which reduces the efficiency of electric stimulation by creating a zone with ionic depletion around the contacts. It would make sense to post-surgically introduce some medicine into the cochlea to maintain a lower electrode impedance. It has been demonstrated, for example, that the introduction of corticosteroids can reduce the impedance rise after surgery. This has been done by depositing or rubbing the medicine on the electrode. But as the electrode is introduced in the fluid of the scala tympani, the medical solution quickly dissolves and may not reach a location where it would be most beneficial or for the desired time when the drug is required post surgically.

There have been attempts with non-cochlear implant patients to deliver medicine to the inner ear for the treatment of Meniere's disease or vertigo. The drug delivery takes place through the somewhat permeable round window membrane after injection of a bolus into the middle ear. One problem with round window drug delivery is that the membrane permeability to molecular substances changes over the course of a day, also large molecules cannot pass through the tight membrane. It is thought that the very little pharmacologic substance reaches to the cochlear region beyond the first few millimeters of cochlea length.

There is no easy existing way to deliver medicine into the inner ear after cochlear implantation. The middle ear is not easily accessed and the inner ear is a sealed system that does not allow direct deposition or injection of medicines except at the time of cochlear implant surgery. After surgery the cochlea is partially filled with the electrode which should not be moved or removed.

Drug eluting electrode leads with corticosteroids have been used successfully in the past with cardiac pacemaker electrodes to reduce the contact impedance. In addition, silicone elastomer loaded with a pharmacologically active agent has been used as an eluting structure in several applications such as contraception, vascular injury treatment, and stents. Drug eluting electrodes have not been used with cochlear implants.

### SUMMARY OF THE INVENTION

Embodiments of the present invention are directed to a cochlear electrode array for electrically stimulating cochlear tissue. The array includes a drug eluting portion adapted to release a therapeutically effective amount of a pharmaceutical agent over time in the inner ear.

In further embodiments, the electrode array may include a slot containing the matched-in-shape drug releasing device, in which case, the geometry of the device may determine the rate at which the pharmaceutical agent is released. The pharmaceutical agent releasing device may be a gel, particulate or solid. The drug eluting portion may be a polymer material such as a silicone based elastomer which incorporates the pharmaceutical agent.

In various embodiments, the drug eluting portion may be a layer of material sandwiched between two layers of non-drug eluting material. For example, the drug eluting portion may constitute 0.25 to 2% of the mass of the electrode array. The drug eluting portion may be embedded within non-drug eluting material so that the thickness of the non-drug eluting material determines the rate at which the pharmaceutical agent will be released. The drug eluting portion may begin at 3 mm or less from where the electrode array enters the inner ear. The release rate of the pharmaceutical agent may be determined by one or more of the crosslink density of the material in the drug eluting and non drug eluting portion, the amount of surface area of the drug eluting portion which is exposed to the non drug eluting sandwich, and the volume of the drug eluting portion.

In some embodiments, the drug eluting portion may include first and second drug eluting portions, each portion adapted to release a different pharmaceutical agent. The electrode array may include multiple electrical contacts for electrically stimulating the cochlear tissue, at least one of the contacts being coated with the pharmaceutical agent. The pharmaceutical agent may be in the form of solid particles of less than 100 μm mixed into the material of the drug eluting portion.

The release rate of the pharmaceutical agent may be based on having particles of the pharmaceutical agent in a plurality of defined sizes. For example, at least 90% of the particles maybe less than 50 μm, and/or at least 50% of the particles maybe less than 10 μm.

The pharmaceutical agent may be a corticosteroid such as betamethasone, clobethasole, diflorasone, fluocinolone, triamcinolone, salt, ester, or combination thereof. Or, the corticosteroid maybe dexamethasone, for example, the electrode array maybe adapted to release between 0.1 μg and 1 μg of dexamethasone during an initial 24 hour period of use.

In some embodiments, the pharmaceutical agent may be an anti-inflammatory agent. For example, the saturated solubility in normal saline of the anti inflammatory agent may be not less than 80 μg/ml at 37° C. The electrode array may be adapted to release between 1 μg and 5 μg of anti inflammatory agent during the first week after implantation. The pharmaceutical agent could be an antibiotic, antioxidant or growth factor.

### BRIEF DESCRIPTION OF THE DRAWINGS

FIG. 1A-F shows various ways to partially load an implanted cochlear electrode with drug eluting silicone.

FIG. 2A-D shows further various specific embodiments of a cochlear electrode with drug eluting silicone.

FIG. 3 shows an embodiment having drug eluting silicone and drug eluting silicone rod in a slot on the electrode.

FIG. 4A-B shows alternative embodiments for incorporating drug eluting silicone with the electrode.

### DETAILED DESCRIPTION OF SPECIFIC EMBODIMENTS

A cochlear electrode array is needed that would allow the release of a therapeutically effective amount of a pharmacological agent for a period of time after surgery. Embodiments of the present invention include a cochlear electrode array based on the incorporation of a given amount of medicine into a portion or whole of the silicone polymer elastomer that makes up the electrode body. Over time, the medicine is released from the elastomeric material and diffused into the fluid of the inner ear. The diffused molecules then target receptors of interest.

The inner ear presents various considerations for localized delivery of pharmacological agents which include being a deep compartment, which means delayed drug action after systemic administration hence, suitable for delivery of antibiotics, corticosteroids, antioxidants and growth factors to regenerate the hearing organ such as neural tissue and soft tissue. The inner ear is a very small and essentially closed space so that any medicine released within the inner ear tends to remain confined within that space however, the pharmacokinetic properties of this organ is not well known. Thus, any pharmacological agent that is slowly released in this environment tends to be bioactive only in the inner ear and there is very little diffusion outside of the inner ear.

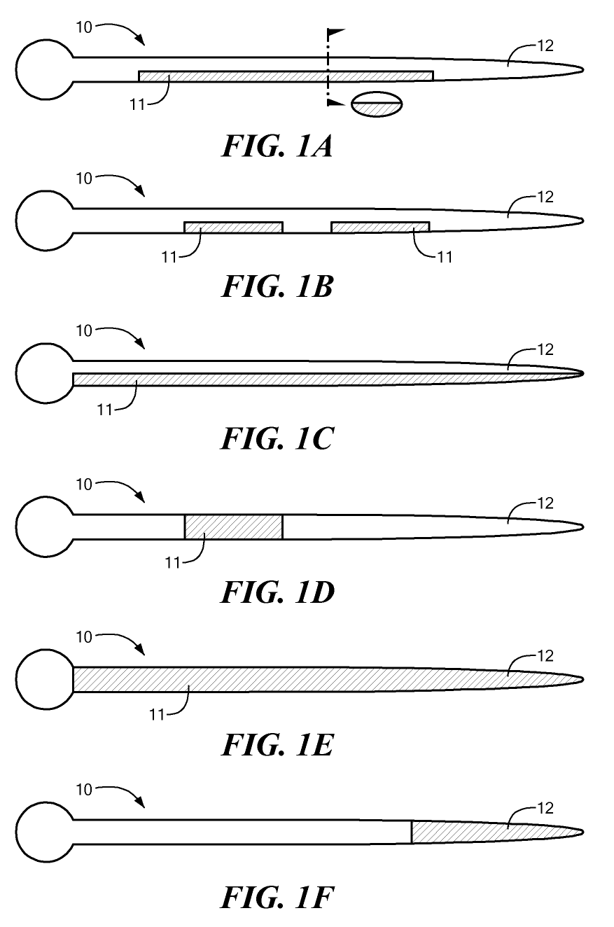

FIG. 1 shows examples of cochlear implant electrode arrays 10 structured so as to include a drug eluting portion 11 and a non-drug eluting portion 12 according to various embodiments of the present invention. In each of the examples shown in FIG. 1, the cross-hatched region represents material adapted to release pharmacological agent, i.e., the drug eluting portion 11. The unshaded regions in FIG. 1 represent material without drug eluting functionality, i.e., the non-drug eluting portion 12.

As shown in FIG. 1A, a cross-section of the electrode array 10 may typically be elliptical or oval in shape. FIG. 1A shows an embodiment in which the lower half of a portion of the electrode array 10 includes is the drug eluting portion 11 including drug eluting material which time releases a pharmacological agent to the surrounding fluid of the inner ear. The upper half of this embodiment is the non-drug eluting portion 12 containing material without drug eluting functionality. FIG. 1B shows another embodiment of an electrode array 10 having two different drug eluting portions 11, each of which may be adapted to release a different pharmacological agent. In the embodiment shown in FIG. 1C, the drug eluting portion 11 includes the entire lower half of the electrode array 10. In such an embodiment, the other structural elements of the electrode array 10 such as the electrical stimulating contacts and connecting wires may be contained within the non-drug eluting portion 12 of the array. In the embodiment shown in FIG. 1D, the entire cross-sectional area of a portion of electrode array 10 is the drug eluting portion 11 which is adapted to incorporate into its material the pharmacological agent for timed release. In FIG. 1E, the entire electrode array 10 uses material incorporating the pharmacological agent. In such an arrangement, the concentration of the pharmacological agent in the elastomeric material may be lower than in embodiments in which a smaller volume portion of the array is used. FIG. 1F shows yet another embodiment in which the entire volume of the forward most portion of the electrode array 10 is adapted to serve as the drug eluting portion 11. For example, the drug eluting portion 11 may begin at 3 mm or more from where the electrode array 10 enters the inner ear.

The rate at which the pharmacological agent is released from the polymer matrix material of the drug eluting portion 11 of the electrode array 10 depends on various factors. These include the amount of surface area of the drug eluting portion 11 which is exposed to the fluid surrounding the polymer or the non loaded polymer. The concentration of medicine within the polymer material of the drug eluting portion 11 also affects the duration of the delivery. The release rate of the pharmacological agent may also depend on other factors such as the crosslink density of the material in the drug eluting portion 11 also the volume of the drug eluting portion 11.

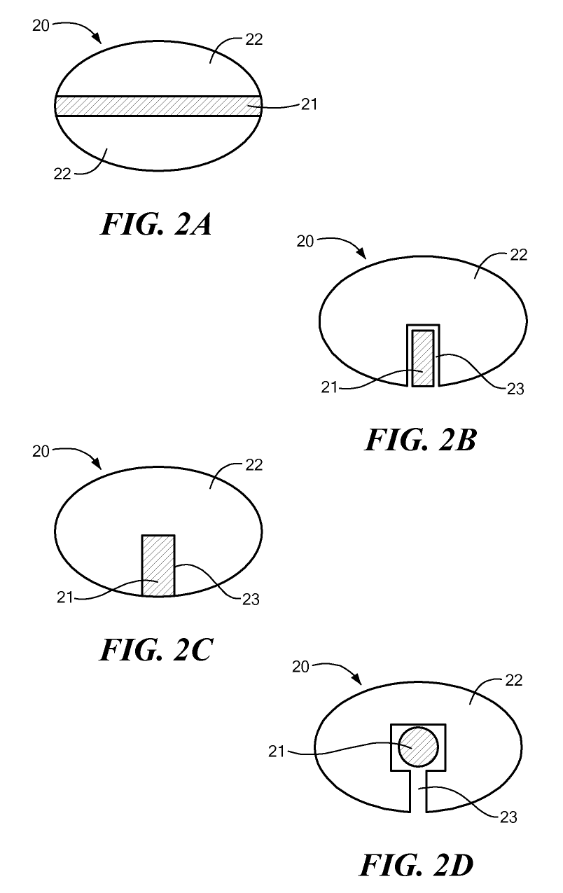

FIG. 2 shows cross-section views of further various embodiments of the present invention. In the example shown in FIG. 2A, the electrode array 20 includes a drug eluting portion 21 which is a layer of material sandwiched between two layers of non-drug eluting material 22. In such an embodiment, the release rate of the pharmacological agent in the drug eluting portion 21 depends on the amount of surface area of the drug eluting portion which is exposed at the sides of the electrode array 20. For example, the mass of the drug eluting portion 21 may constitute 0.25% to 2% of the mass of the electrode array 20.

In the embodiments shown in FIGS. 2B-D, the electrode array 20 includes a channel slot 23 in the non-drug eluting material 22 into which the material of the drug eluting portion 21 is incorporated. In FIG. 2B, the drug eluting portion 21 is in the form of a rod which is slightly smaller than the channel slot 23 holding it, so that the fluid of the inner ear contacts the entire perimeter of the drug eluting portion 21, which over time releases pharmacological agent into the inner ear fluid. In FIG. 2C, the drug eluting portion 21 fits more snugly into the channel slot 23 of the non-drug eluting material 22. Thus, only the bottom surface of the drug eluting portion 21 contacts the fluid of the inner ear so as to release pharmacological agent more slowly. In the embodiment shown in FIG. 2D, a round rod of drug eluting material 21 is embedded in a channel slot 23 in the non-drug eluting material 22 which has a square cross-sectional region that allows controlled access of the inner ear fluid to the surface area of the cylindrical rod of drug eluting material 21.

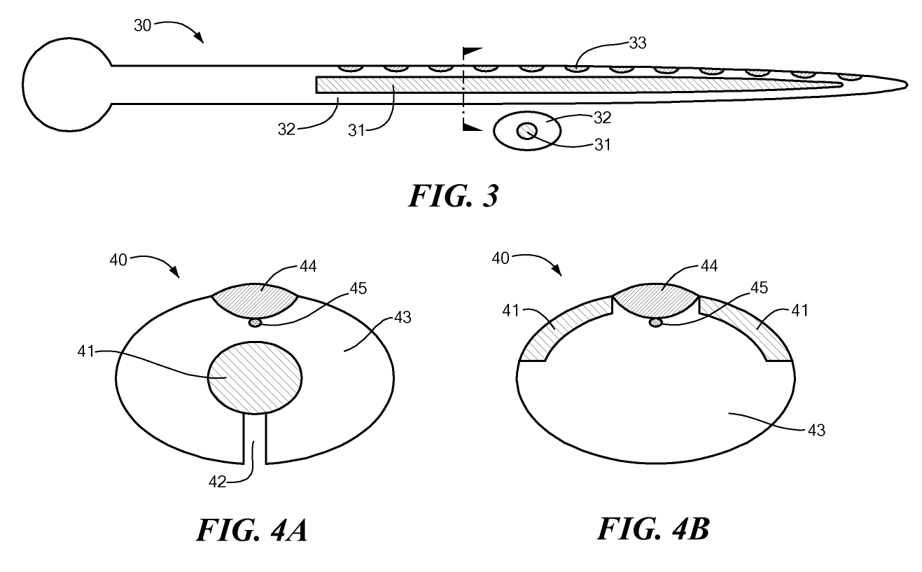

FIG. 3 shows an embodiment of an electrode array 30 (including electrode contacts 33) in which the drug eluting portion 31 is entirely embedded within non-drug eluting material 32. In such an embodiment, the rate at which the pharmacological agent is released by the drug eluting portion 31 is determined by the parameters of the drug eluting portion such as loading and surface area also thickness of the overlying layer of non-drug eluting material 33.

FIG. 4A shows a cross section of another embodiment of an electrode array 40 similar to the one shown in FIG. 3, but also including a channel slot 42 in the non-drug eluting material 43 that allows some of the inner ear fluid to contact a portion of the surface area of the drug eluting portion 41. Again, the release rate of the pharmacological agent is determined by the amount of surface area of the drug eluting portion 41 that is exposed, as well as the concentration of pharmacological agent in the material of the drug eluting portion 41, and possibly the diffusion rate of pharmacological agent through the drug eluting material. FIG. 4B shows another embodiment of an electrode array 40 in which silicon material of the drug eluting portion 41 is disposed on either side of the electrode contacts 44 on the surface of the electrode array 40, with the remainder of the electrode area being neat silicone material. In such an embodiment, one or more of the electrode contacts 44 may also be coated with a pharmaceutical agent.

Examples of specific pharmacological agents suitable for post-surgical release into the inner ear include without limitation neurotrophic factors, gene therapy agents, anti-apoptosis medicines, and anti-oxidants and antibiotics. Some medicines have neuro-protective effects and could help to sustain the neural status of the inner ear after the somewhat traumatic cochlear implantation.

Other suitable pharmacological agents include anti inflammatory agents. These hydrophobic and sparingly soluble agents may help to overcome the local inflammation after cochlear implantation surgery. For example, the saturated solubility in normal saline of the anti inflammatory agent may be 80 μg/ml at 37 C.°. The electrode array may be adapted to release less than 1 μg to 5 μg of anti inflammatory agent during the first week after implantation. The device may also deliver other agents such as one or more of a bactericide, antibiotic, antioxidant, or growth factor in parallel with the cortico steroid using the proposed designs as mentioned above with two distinct drug loaded region (FIGS. 1-B and 4-B).

Of special and immediate interest is the use of corticosteroids to control post-implantation fibrotic development. One example of such a corticosteroid is dexamethasone. For example, the electrode array may be adapted to release between 0.1 and 1 μg of dexamethasone during an initial 24 hour period of use. Other examples of corticosteroids suitable for use in a drug eluting cochlear implant electrode array include without limitation betamethasone, clobethasole, diflorasone, fluocinolone, triamcinolone, or salt, ester, or combination thereof.

Due to low solubility of the corticosteroids; a silicone-based drug eluting device can be produced by first micronizing the pharmaceutical agent particles to a desired size. For example, the pharmaceutical agent may be in the form of solid particles of less than 100 μm mixed into the material to prepare the drug eluting portion. The release rate of the pharmaceutical agent may be based on having particles of the pharmaceutical agent in a plurality of defined sizes. For example, in some embodiments, at least 90% of the particles may be less than 50 μm in size. In addition or alternatively, at least 50% of the particles may be less than 10 μm in size. The particles can be thoroughly mixed in a validated way with liquid silicone polymer using a high speed dual centrifugal mixer. In all embodiments, a cross-linking solution may be added to the mixture. The resulting mixture is then injected into the space reserved for the drug eluting portion using a properly designed mold.

Concentration of the pharmaceutical agent in the surrounding inner ear fluid depends on the drug loading and permeability of the pharmaceutical agent in the drug eluting material. The release time may be days to months depending on the crosslinking density of the silicone, amount of loading of drug as a percentage of electrode array, volume of drug loaded polymer, and surface area exposed to the fluid of the cochlea.

An electrode array according to an embodiment of the invention can be assembled in various steps. For example, the wires and electrode contacts used for electrical stimulation can be placed in one half of an array mold. A first stage of molding then encapsulates the wires and electrode contacts using a reverse molding or masking to leave a space where the drug eluting silicone material can be injected in a second step. This approach allows bonding of the two similar polymers to ensure a uniform contour of the electrode.

One advantage of using a two-stage molding process is that only a portion of the electrode array in the fluid of the inner ear need be loaded with a pharmaceutical agent. The extra cochlea portion of the electrode array can be made of non-drug eluting material and need not participate in the drug release.

A multi-stage molding process involving multiple masking can also be used to successively add complimentary drug eluting material in more than one place, with each drug eluting portion having a different composition of pharmaceutical agent. In this manner, complimentary drugs or drugs targeting different receptors and at a different rate of diffusion can be incorporated in the electrode array.

Polymer rods loaded with a pharmacologically active agent may be prefabricated. The rod of drug eluting material may be made of a silicone of the same or similar composition as that used in the fabrication of the main non-drug eluting portion of the electrode array. For example, drug eluting rods can be prefabricated in a high level pharmaceutical lab equipped with the necessary instrumentation. The rods can then be shipped to be assembled with the cochlear implant electrode array at another location. For example, the electrode arrays shown in FIGS. 2B, 2D, and 4 could be prefabricated for final assembly with prefabricated drug eluting rod.

Although various exemplary embodiments of the invention have been disclosed, it should be apparent to those skilled in the art that various changes and modifications can be made which will achieve some of the advantages of the invention without departing from the true scope of the invention.

What is claimed is:

1. A cochlear implant electrode array comprising:
a cochlear electrode array for electrically stimulating cochlear tissue, the array including a drug eluting portion adapted to release over time a therapeutically effective amount of a pharmaceutical agent for the inner ear.

2. An electrode array according to claim 1, wherein the electrode array includes a slot containing a rod loaded with a pharmaceutical agent.

3. An electrode array according to claim 2, wherein the geometry of the slot determines the rate at which the pharmaceutical agent is released.

4. An electrode array according to claim 1, wherein the pharmaceutical agent is a gel, particulate or solid.

5. An electrode array according to claim 1, wherein the drug eluting portion is a polymer material incorporating the pharmaceutical agent.

6. An electrode array according to claim 5, wherein the polymer material is a silicon-based elastomer.

7. An electrode array according to claim 1, wherein the drug eluting portion is a layer of material sandwiched between two layers of non-drug eluting material.

8. An electrode array according to claim 7, wherein the drug eluting portion comprises 0.25% to 2% of the mass of the electrode array.

9. An electrode array according to claim 1, wherein the drug eluting portion is embedded within non-drug eluting material.

10. An electrode array according to claim 9, wherein the thickness of the non-drug eluting material determines the rate at which the pharmaceutical agent is released.

11. An electrode array according to claim 1, wherein the drug eluting portion begins at 3 mm or less from where the electrode array enters the inner ear.

12. An electrode array according to claim 1, wherein the release rate of the pharmaceutical agent is based on cross-link density of the material in the drug eluting portion.

13. An electrode array according to claim 1, wherein the release rate of the pharmaceutical agent is based on the amount of surface area of the drug eluting portion which is exposed to the fluid of the inner ear.

14. An electrode array according to claim 1, wherein the release rate of the pharmaceutical agent is based on the volume of the drug eluting portion.

15. An electrode array according to claim 1, wherein the drug eluting portion includes first and second drug eluting portions, each portion adapted to release a different pharmaceutical agent.

16. An electrode array according to claim 1, wherein the electrode array includes a plurality of electrical contacts for electrically stimulating the cochlear tissue, at least one of the contacts being coated with the pharmaceutical agent.

17. An electrode array according to claim 1, wherein the pharmaceutical agent is in the form of solid particles of less than 100 μm mixed into the material of the drug eluting portion.

18. An electrode array according to claim 1, wherein the release rate of the pharmaceutical agent is based on having particles of the pharmaceutical agent in a plurality of defined sizes.

19. An electrode array according to claim 18, wherein at least 90% of the particles are less than 50 μm.

20. An electrode array according to claim 18, wherein at least 50% of the particles are less than 10 μm.

21. An electrode array according to claim 1, wherein the pharmaceutical agent is a corticosteroid.

22. An electrode array according to claim 21, wherein the corticosteroid includes betamethasone, clobethasole, diflorasone, fluocinolone, triamcinolone, salt, ester, or combination thereof.

23. An electrode array according to claim 21, wherein the corticosteroid is dexamethasone.

24. An electrode array according to claim 23, wherein the electrode array is adapted to release between 0.1 and 1 μg of dexamethasone during an initial 24 hour period of use.

25. An electrode array according to claim 1, wherein the pharmaceutical agent is an anti-inflammatory agent.

26. An electrode array according to claim 25, wherein the saturated solubility in normal saline of the anti inflammatory agent is not less than 80 μg/ml at 37° C.

27. An electrode array according to claim 25, wherein the electrode array is adapted to release between 1 μg and 5 μg of anti inflammatory agent during the first week after implantation.

28. An electrode array according to claim 1, wherein the pharmaceutical agent is an antibiotic, antioxidant, or growth factor.
